(19) United States
(12) Patent Application Publication
(10) Pub. No.: US 2022/0323204 A1
(43) Pub. Date: Oct. 13, 2022

(54) METHOD OF MANUFACTURING AN OCULAR PROSTHESIS AND OCULAR PROSTHESIS THEREOF

(71) Applicant: Ocupeye Limited, Warwickshire (GB)

(72) Inventors: Stephen Geoffrey Bell, London (GB); Gordon James Bott, Oxfordshire (GB)

(73) Assignee: Ocupeye Limited, Warwickshire (GB)

(21) Appl. No.: 17/639,219

(22) PCT Filed: Aug. 28, 2020
(86) PCT No.: PCT/GB2020/052070

§ 371 Date: Feb. 28, 2022

(30) Foreign Application Priority Data
Aug. 30, 2019 (GB) 1912438.7

(51) Int. Cl. A61F 2/14; B33Y 50/00; B29D 11/02; B29C 64/386; B33Y 80/00

(52) U.S. Cl. CPC A61F 2/141; B33Y 50/00; B29D 11/023; B29C 64/386; B33Y 80/00; A61F 2240/002; A61F 2210/0076; A61B 3/102

(57) ABSTRACT

A method (100) of forming an ocular prosthesis (13) comprising: receiving (102a) three dimensional scan data of a patients eye, the three dimensional scan data including volumetric data of at least an iris region, a pupil region and a cornea region of the eye; and forming (110) an ocular prosthesis (13) by an additive manufacturing process, the ocular prosthesis (13) having a body (15) with a volume, wherein the body of the prosthesis includes a three dimensional iris region (23), a three dimensional pupil region (25) and a three dimensional cornea region (26), and the volumetric data determines the size and shape of the iris region (23), the pupil region (25) and the cornea region (26) of the prosthesis (13).

The present disclosure relates to ocular prostheses, prosthetic eyes, and methods of manufacturing ocular prostheses.

Ocular prostheses may be worn by anophthalmic patients (patients who have lost an eye, because of a disease, congenital defect or trauma or microphthalmic patients (patients with an eye that is abnormally small, due to a birth defect).

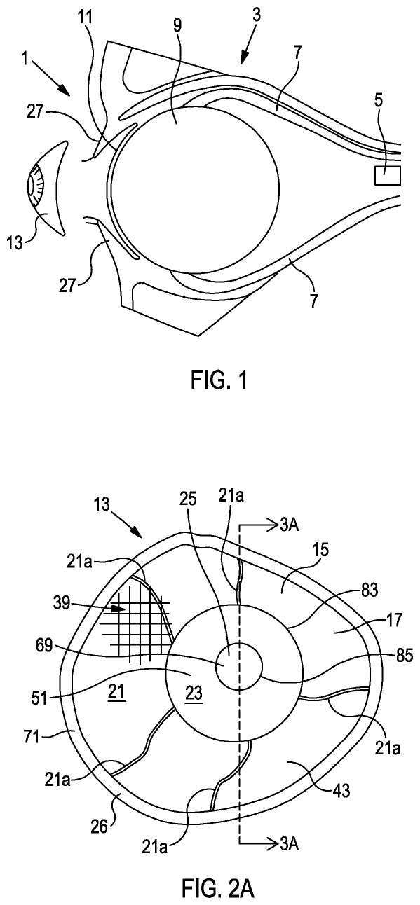

FIG. 1 schematically illustrates an example of a prosthetic eye 1 in an anophthalmic eye socket 3. When the patient's eye is removed, the optic nerve 5 is severed, and the eyeball is detached from the muscles 7 which control its movement. After the eyeball is taken out of the socket 3, the patient is then fitted with an orbital implant 9, to which the muscles 7 are reattached. The implant 9 is covered with the conjunctiva skin layer 11, and when the swelling from the surgery dies down, an ocular prosthesis 13 is provided. The ocular prosthesis 13 should match the aesthetic appearance of the real eye, and is removable, for regular cleaning. For a microphthalmic patient, the ocular prosthesis 13 is worn with the existing eyeball, rather than the orbital implant 9. In either case, the ocular prosthesis 13 is custom made for both size and appearance.

The prosthesis 13 should form a loose friction fit with the implant 9 or microphthalmic eye. Thus, when the implant 9 or microphthalmic eye is moved by the muscles 7, the prosthesis 13 should move in a corresponding manner. However, the friction fit should also provide for sufficient oxygenation of the eye socket.

Currently, the process for making an ocular prosthesis 13 involves making an impression of the anophthalmic socket or microphthalmic eye and surrounding tissue using an impression material. The body of the prosthesis 13 is then moulded by hand using the impression. A separate disc forming the iris is made, and attached to the moulded body. A transparent cornea is then over moulded on top of the body.

The shape of the rear surface of the ocular prosthesis is based on the impression. The rest of the shape of the prosthesis is determined based on standard parts. Both the iris and the sclera are hand painted whilst the patient is present in clinic to provide the desired appearance.

The process of taking the impression can be uncomfortable and difficult for both the patient and the occularist taking the impression. In addition, because of these difficulties in the process of taking the impression, the impression may be inaccurate. This may cause a poor fit of the prosthesis 13 to the implant 9 or microphthalmic eye, such that the prosthesis requires modification or, is uncomfortable and/or does not move as it should.

Furthermore, the process from taking the impression to completing the eye can take several weeks or more. Younger patients may need to repeat the process on a regular basis to be fitted for new prostheses as they grow.

According to a first aspect of the invention, there is provided a method of forming an ocular prosthesis comprising: receiving three dimensional scan data of a patient's eye, the three dimensional scan data including volumetric data of at least an iris region, a pupil region and a cornea region of the eye; and forming an ocular prosthesis by an additive manufacturing process, the ocular prosthesis having a body with a volume, wherein the body of the prosthesis includes a three dimensional iris region, a three dimensional pupil region and a three dimensional cornea region, and the volumetric data determines the size and shape of the iris region, the pupil region and the cornea region of the prosthesis.

The body may a front surface opposing the rear surface, and a thickness between the front surface and the rear surface.

The shape of a front surface of the prosthesis may be determined by the three dimensional scan data.

The method may further comprise receiving second three dimensional scan data of a fitting surface on which the ocular prosthetic is to be fitted. A rear surface of the prosthesis may be formed so as to confirm to the fitting surface.

The method may further comprise receiving image data of the patient's eye, the image data including colour and pattern information, and wherein a pattern of the different regions of the prosthesis varies through the thickness of the volume based on the colour and pattern information.

Forming an ocular prosthesis by an additive manufacturing process may comprise varying one or more properties through the thickness of the body.

The one or more properties may comprise one or more of: a colour and/or pattern of the body; and a hardness of the body.

The body may be formed by depositing a material forming the body on a support.

The colour of the material may be changed by varying a pigment added to the material of the body as it is.

The hardness of the material may be changed by varying the relative proportion of an additive included in the material of the body as it is deposited.

The method may further comprise forming one or more channels for directing tear flow to a rear surface of the body, during the additive manufacturing process.

The one or more channels may be formed as grooves in the rear surface of the body or the one or more channels may be formed as passages. At least part of the passages may be formed within the body, with openings on the rear surface.

The method may further comprise forming one or more voids in the body, during the additive manufacturing process.

The method may further comprise forming an outer shell on an exterior of the body, by the additive manufacturing process.

The method may further comprise following completion of the additive manufacturing process, forming a coating on the body, the coating having a thickness of 10 microns or less.

The coating may be hydrophilic.

A material of the body may be varied through the prosthesis.

The body may comprise VERO. For example, the body may comprise one or more VERO materials, on their own or in a mixture. The VERO materials may include: VERO Cyan, VERO Magenta, VERO Yellow, VERO Black, VERO White, VERO clear.

The additive manufacturing process may comprise a voxel printing process. The additive manufacturing process may comprise polyjet printing.

According to an second aspect of the invention, there is provided a method of manufacturing an ocular prosthesis comprising: scanning an anophthalmic or microphthalmic eye socket in order to measure a three dimensional fitting surface on which an ocular prosthetic is to be fitted; and manufacturing an ocular prosthesis by an additive manufacturing process, the prosthesis having a rear surface arranged to conform to the scanned fitting surface.

The method may comprise: scanning an anterior chamber of a patient's eye; analysing the scan to determine the size and shape of the sclera, iris, pupil and cornea of the companion eye; and forming the ocular prosthesis with sclera, iris, pupil and cornea regions corresponding to the scanned eye.

The method may comprise: combining the scanned surface of the anophthalmic or microphthalmic eye socket with the size and shape of the sclera, iris, pupil and cornea of the scanned eye to form a schematic of the prosthesis; and manufacturing the prosthesis according to the schematic.

The method may comprise: an image of the patient's eye; analysing the image to determine a colour and pattern of the sclera and iris region; and forming the ocular prosthesis with a sclera and iris region corresponding to the companion eye.

The colour and pattern of the sclera and iris region may be incorporated into the schematic.

The two dimensional image may be captured in controlled lighting conditions.

The image and scan of the patient's eye may be taken consecutively, using a combined scanner and photography system.

Random variations may be introduced into schematic prior to manufacture.

The patient's eye may be either a companion eye to the anophthalmic or microphtalmic eye socket, or an eye in the anophthalmic or microphtalmic socket prior to removal.

Scanning an anophthalmic or microphthalmic eye socket may comprises optical coherence tomography. Scanning a corresponding companion eye may comprise optical coherence tomography.

The optical coherence tomography scan may comprise a plurality of linear scans extending diametrically across the eye socket, the linear scans arranged around an axis extending into the eye socket. Scanning the anophthalmic or microphthalmic eye socket and/or scanning the companion eye may comprise taking a plurality of linear scans around the axis.

The scan may measure point data spaced by a first spacing. The method may include: converting the scan to have point data spaced by a different spacing.

The method may include: auto-focussing the scan on a temporary prosthesis received in the anophthalmic or microphthalmic eye socket, wherein the temporary prosthesis includes an auto-focussing point spaced from the surface.

Using optical coherence tomography is a non-invasive and comfortable method for measuring the patient for the prosthesis, and also provides an accurate scan to make a well fitted prosthesis. Furthermore, the time taken to take a scan is less than the time taking to make an impression of the implant and surrounding tissue.

The use of a colour image also allows for more accurate matching of colours, to provide a more realistic appearance. By capturing the image in controlled lighting conditions, the colours of the prosthetic can be calibrated so that they better match the true colour of the companion eye.

Manufacturing an ocular prosthesis by an additive manufacturing process in the second aspect may comprise the method of the first aspect.

According to a third aspect of the invention, there is provided an ocular prosthesis made according to the first or second aspect.

According to a fourth aspect of the invention, there is provided an ocular prosthesis comprising: a body having a rear surface, and a front surface opposing the rear surface, and a thickness defined between the front surface and rear surface, wherein the body includes two or more three dimensional regions formed continuously with each other by an additive manufacturing process, each region having a thickness forming at least part of the thickness of the body and a cross-sectional area extending perpendicular to the thickness of the body.

A first three dimensional region may comprise an iris region, and a second three dimensional region may comprise a pupil region surrounded by the iris region. The pupil region may be formed by a dark pigment or ink added to the material forming the body. The pupil region may be formed within a recess defined in the iris region.

The recess may widen towards the rear surface, such that the iris region overlies a peripheral portion of the pupil region in the direction of the thickness.

The ocular prosthesis my further include a cornea region forming the front surface of the body, wherein the cornea region fills at least part of the recess in the iris region.

The ocular prosthesis my further include a sclera region, the sclera region including a first sub-region defining a background colour or pattern, and one or more second sub-regions defining veins. The first sub-region may comprise a front facing surface, wherein the one or more second sub-regions may be provided at any one or more of the following positions:
forward of the front facing surface, such that the second sub-regions form bumps on the surface of the sclera region; and/or
at the front facing surface, such that the second sub-regions are partially in front of the front facing surface and partially behind the front facing surface, and form bumps on the surface of the sclera region; and/or
behind the front facing surface.

According to a fifth aspect of the invention, there is provided an ocular prosthesis comprising: a body having a rear surface, and a front surface opposing the rear surface, and a thickness defined between the front surface and rear surface; and an outer shell at least partially encasing the body, wherein at least the outer shell is formed of a biocompatible material.

The body may comprise a non-biocompatible material, and the coating completely encases the body.

The outer shell may have a thickness of between 200 and 500 microns.

The outer shell may be formed continuously with the body, such that the body and outer shell are a single continuous structure.

The body may include a plurality of layers formed between the front surface and the rear surface and the shell is formed on an exterior part of each layer.

The outer shell may comprise a different material to the body.

The outer shell may be biocompatible, and the body is non-biocompatible.

The outer shell may be transparent.

The outer shell may comprise a core and an outer coating forming an exterior of the ocular prosthesis.

The outer coating may be 10 microns thick at most.

The coating may comprise a wetting treatment to make an outer surface of the body hydrophilic.

The body may comprise a sclera region, an iris region, a pupil region, and a cornea region encased within the outer shell.

The body may comprise VERO. For example, the body may comprise one or more VERO materials, on their own or in a mixture. The VERO materials may include: VERO Cyan, VERO Magenta, VERO Yellow, VERO Black, VERO White, VERO clear.

The body and outer shell comprise a plurality of voxels.

The body may include one or more channels for directing tearflow to the rear surface of the body. The one or more channels may be formed as recesses in the rear surface of the body. The one or more channels may alternatively be formed as passages. At least part of the passages may be formed within the body, with openings on the rear surface.

According to a sixth aspect of the invention, there is provided an ocular prosthesis comprising: a body having a rear surface, and a front surface opposing the rear surface, and a thickness defined between the front surface and rear surface, wherein a hardness of a material forming the body is varied through the thickness of the body.

The hardness of the material may be relatively lower towards the rear surface.

The body may include one or more channels for directing tearflow over at least part of the rear surface of the body.

The rear surface may be arranged to, in use, engage a conjunctiva, and the one or more channels direct flow to the interface between the conjunctiva and rear surface.

The one or more channels may be arranged to direct tearflow from a lacrimal gland to a lacrimal canal. The ocular prosthesis may include: a first channel arranged to collect tearflow form the lacrimal gland, and provide tearflow to the rear surface; and a second channel arranged to collect tearflow from the rear surface and carry the tearflow to the lacrimal canal.

The one or more channels may be formed as recesses in the rear surface of the body,

The one or more channels are formed as passages. At least part of the passages may be formed within the body, with openings on the rear surface.

A hardness of the material may be relatively higher towards the front surface.

The hardness of the material may be varied by altering a material composition of the material through the body.

The body may include two or more three dimensional regions, each region having a thickness forming at least part of the thickness of the body and a cross-sectional area extending perpendicular to the thickness of the body. The two or more regions may be formed continuously with each other such that the body is a single continuous structure. A pattern of at least one of the regions may vary through the thickness of the body.

The body may comprise VERO. For example, the body may comprise one or more VERO materials, on their own or in a mixture. The VERO materials may include: VERO Cyan, VERO Magenta, VERO Yellow, VERO Black, VERO White, VERO clear.

The body may comprise a plurality of voxels, and wherein the hardness of the voxels are varied through the body, to vary the hardness of the regions.

The ocular prosthesis may comprise one or more voids within the body.

The body may comprise an outer shell comprising a biocompatible material.

The ocular prosthesis may comprise a coating encasing the body, the coating having a thickness less than 10 microns. The coating may comprise a wetting treatment to make an outer surface of the body hydrophilic. The coating may comprise a plasma coating.

According to a seventh aspect of the invention, there is provided an ocular prosthesis comprising: a body having a rear surface, and a front surface opposing the rear surface, and a thickness defined between the front surface and rear surface; and one or more channels formed in the body, arranged to direct tearflow to the rear surface of the body.

The rear surface may be arranged to, in use, engage a conjunctiva. The one or more channels may be arranged to direct tearflow to the interface between the conjunctiva and rear surface.

The one or more channels may be arranged to direct tearflow from a lacrimal gland to a lacrimal canal.

The ocular prosthesis may include: a first channel arranged to collect tearflow form the lacrimal gland, and provide tearflow to the rear surface; and a second channel arranged to collect tearflow from the rear surface and carry the tearflow to the lacrimal canal.

The one or more channels may be formed as recesses in the rear surface of the body,

The one or more channels may alternatively be formed as passages. At least part of the passages may be formed within the body, with openings on the rear surface.

The one or more channels may be arranged to directing tearflow over at least part of the rear surface of the body.

The body may comprise VERO. For example, the body may comprise one or more VERO materials, on their own or in a mixture. The VERO materials may include: VERO Cyan, VERO Magenta, VERO Yellow, VERO Black, VERO White, VERO clear.

The body may comprise a plurality of voxels. The properties of the voxels may be varied through the body, to vary the material properties of the body. The colour of the voxels may be varied through the body, to vary the pattern of the regions.

The ocular prosthesis may comprise one or more voids within the body.

According to a eighth aspect of the invention, there is provided an ocular prosthesis comprising: a body having a rear surface, and a front surface opposing the rear surface, and a thickness defined between the front surface and rear surface; and one or more voids formed within the body.

The voids may be formed into the thickness of the body, away from the front and rear surfaces, such that they do not extend onto the front surface or rear surface of the body.

The body may include two or more three dimensional regions, each region having a thickness forming at least part of the thickness of the body and a cross-sectional area extending perpendicular to the thickness of the body.

The voids may be formed in a first region only, the first region forming at least part of the rear surface.

The voids may be formed sufficiently far from the front surface, into the thickness, such that they are not visible on the front surface.

The two or more regions may be formed continuously with each other such that the body is a single continuous structure.

A pattern of at least one of the regions may vary through the thickness of the body.

A hardness of a material forming the body may be varied through the thickness of the body. The hardness of the material may be reduced towards the rear surface. The hardness of the material may be higher towards the front surface.

The ocular prosthesis may comprise one or more channels formed in the body, arranged to direct tearflow to the rear surface of the body.

The ocular prosthesis may comprise an outer shell at least partially encasing the body. At least the outer shell may be formed of a biocompatible material.

The body may comprise VERO. For example, the body may comprise one or more VERO materials, on their own or in a mixture. The VERO materials may include: VERO Cyan, VERO Magenta, VERO Yellow, VERO Black, VERO White, VERO clear.

The body may comprise a plurality of voxels.

According to a ninth aspect of the invention, there is provided an ocular prosthesis comprising: a body having a rear surface, and a front surface opposing the rear surface, and a thickness defined between the front surface and rear surface, wherein the body includes two or more three dimensional regions, each region having a thickness forming at least part of the thickness of the body and a cross-sectional area extending perpendicular to the thickness of the body; wherein the two or more regions are formed continuously with each other such that the body is a single continuous structure; and wherein a pattern of at least one of the regions varies through the thickness of the body.

The body may be formed by a plurality of layers, each layer extending perpendicular to the thickness of the body. At least some of the layers may include portions of at least two of the two or more regions, the portions being formed continuously with each other within a layer. The layers may be formed continuously with each other.

The body may include one or more layers having a portion of only one of the at least two or more regions.

At least part of one or more region may have a plain pattern. The plain pattern may be provided towards the rear surface of the prosthesis.

The ocular prosthesis may include a transparent region provided towards the front surface. The transparent region may overlie the other regions. The transparent region may extend around the sides of the body, to form at least part of the rear surface.

The cross-sectional area of each region may vary with thickness through the body.

The two or more regions may be selected from the following: a three dimensional iris region; a three dimensional sclera region; a three dimensional pupil region; and a three dimensional cornea region.

The sclera region may include a first sub-region defining a background colour or pattern, and one or more second sub-regions defining veins. The one or more second sub-regions may extend at portion of the thickness of the region.

The pupil region may be surrounded by the iris region. The pupil region may be formed by a dark pigment or ink added to the material forming the body. The pupil region may be formed within a recess defined in the iris region. The recess may widen towards the rear surface, such that the iris region overlies a peripheral portion of the pupil region in the direction of the thickness. The cornea region may fill at least part of the recess in the iris region.

The ocular prosthesis may comprise different materials for forming the different regions.

The patterns may be formed by adding pigment to the material forming the body.

The body may comprise a plurality of voxels. The colour of the voxels may be varied, to form the patterns.

The body may comprise an outer shell comprising a biocompatible material.

The ocular prosthesis may comprise a coating encasing the body, the coating having a thickness less than 10 microns.

In the various aspects of the invention, the body may comprise VERO. For example, the body may comprise one or more VERO materials, on their own or in a mixture. The VERO materials may include: VERO Cyan, VERO Magenta, VERO Yellow, VERO Black, VERO White, VERO clear.

In the various aspects of the invention, the body may comprise comprises a plurality of voxels, and wherein the colour of the voxels is varied through the body.

The various aspects discussed above provide for a prosthesis that mimics the appearance of a human eye in various different ways (bio-mimicking). The body may be considered to be a single unitary structure. Therefore, there is no requirement to form separate components, and later join those components together. For example, the iris, and sclera may be formed as a single unitary structure, integral with the sclera. This makes the manufacture of the prosthesis simpler and faster.

In the various aspects of the invention discussed above, varying the pattern though the thickness of body provides for a more realistic appearance, especially in the iris region of the eye and in the veins on the sclera.

Varying the hardness of the material through the thickness of the body allows the rear surface to be formed in such a manner that it can grip onto the orbital implant. Thus the movement of the prosthesis with the orbital implant (and hence the healthy eye) can be improved.

The provision of channels ensures proper wetting of the prosthesis, and thus lubrication and oxygenation of the eye in the socket. This may be of particular use if the prosthesis is of particularly tight fit to the conjunctiva or microphthalmic eye. The use of channels that direct the flow of tears also stops tears becoming trapped by the prosthesis, reducing the risk of bacterial infection.

The use of a coating provides a variety of possible advantages. It may improve the wetting and lubrication of the prosthetic eye, in the eye socket. Furthermore, it may maintain the prosthesis in a cleaner condition, such that less regular cleaning is required. Also, when the coating fully encases the body, it means that the body of the prosthesis may contain non-biocompatible and/or porous materials.

The formation of voids within the body of the prosthesis reduces the weight of the prosthesis. This may prevent sagging of the prosthesis under gravity, especially where a larger prosthesis is required.

According to a tenth aspect of the invention, there is provided a method of manufacturing the prosthesis of any preceding aspect, the method comprising: forming the body using an additive manufacturing process.

The additive manufacturing process may comprise polyjet printing.

When an outer coating is provided, the method may further comprise: applying the outer coating after forming the body and core of the outer shell by the additive manufacturing process. The coating may be applied by plasma coating.

According to a eleventh aspect of the invention, there is provided a prosthetic eye including: an orbital implant, and an ocular prosthesis according to any of the above aspects of the invention.

The methods according to various aspects allow for a significant reduction in the time taken to manufacture prosthesis. Furthermore, the use of additive manufacturing techniques allows for greater control of the physical and aesthetical properties of the prosthesis, and allows for a much better fitting prosthesis to be made.

It will be appreciated that any features discussed in relation to one of the above aspects may be applied to any other aspect, unless mutually exclusive.

Embodiments of the invention will now be described, by way of example only, with reference to the accompanying drawings, in which:

FIG. 1 generally illustrates a prosthetic eye for an anophthalmic patient;

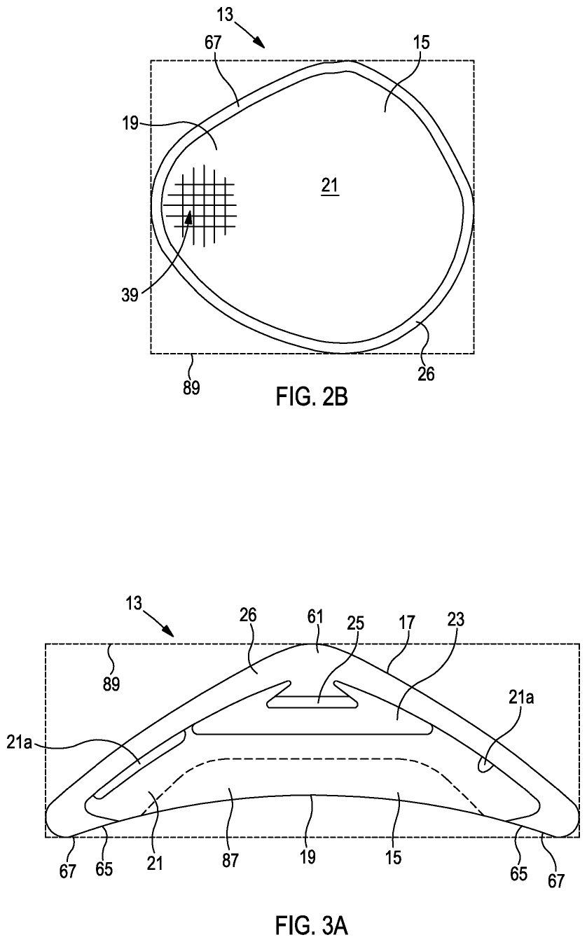

FIG. 2A schematically illustrates a front view of an ocular prosthesis according to an embodiment of the invention;

FIG. 2B schematically illustrates a rear view of the prosthesis shown in FIG. 2A;

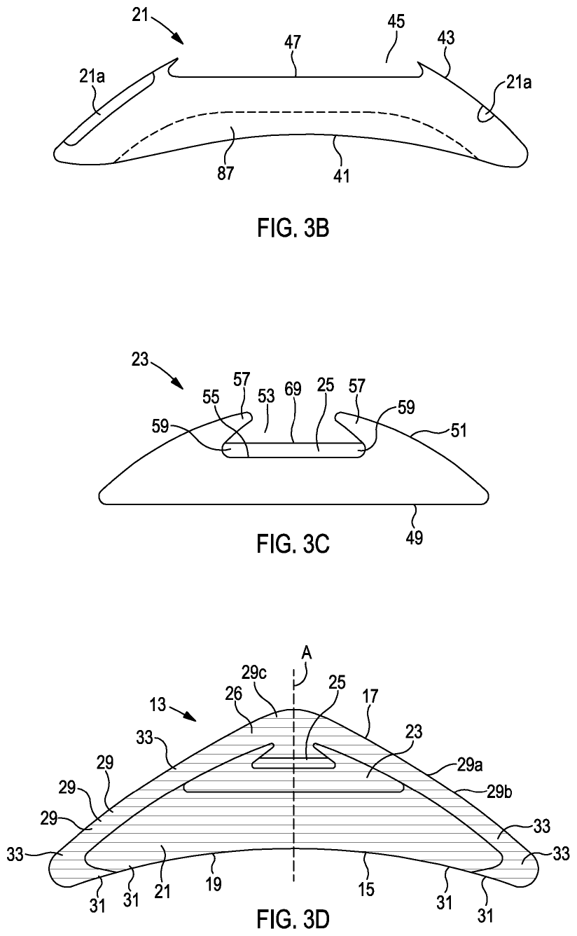

FIG. 3A schematically illustrates a cross section through the prosthesis shown in FIG. 2A;

FIG. 3B schematically illustrates a cross section through the sclera region of the prosthesis of FIG. 2A;

FIG. 3C schematically illustrates a cross section through the iris and pupil regions of the prosthesis of FIG. 2A;

FIG. 3D schematically illustrates a cross section through the prosthesis of FIG. 2A, illustrating the layered structure;

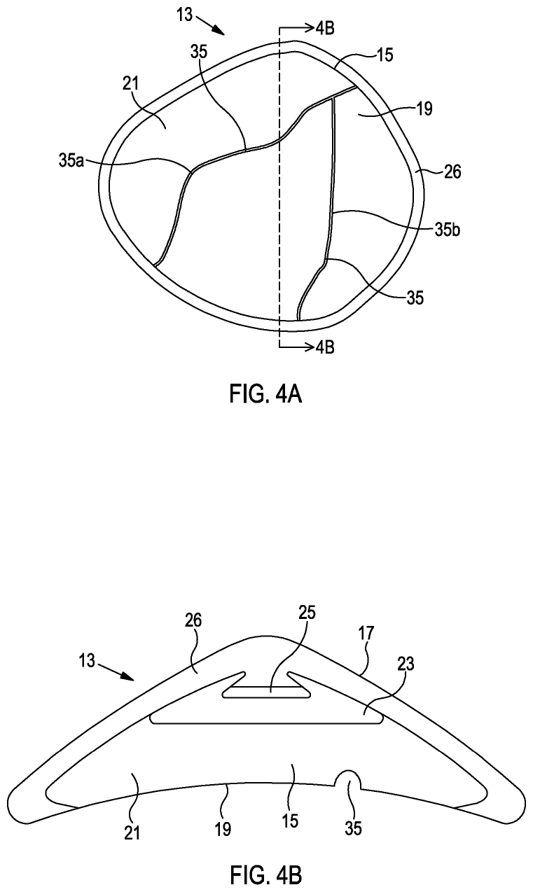

FIG. 4A schematically illustrates a rear view of another example prosthesis;

FIG. 4B schematically illustrates a cross section through the prosthesis shown in FIG. 4A;

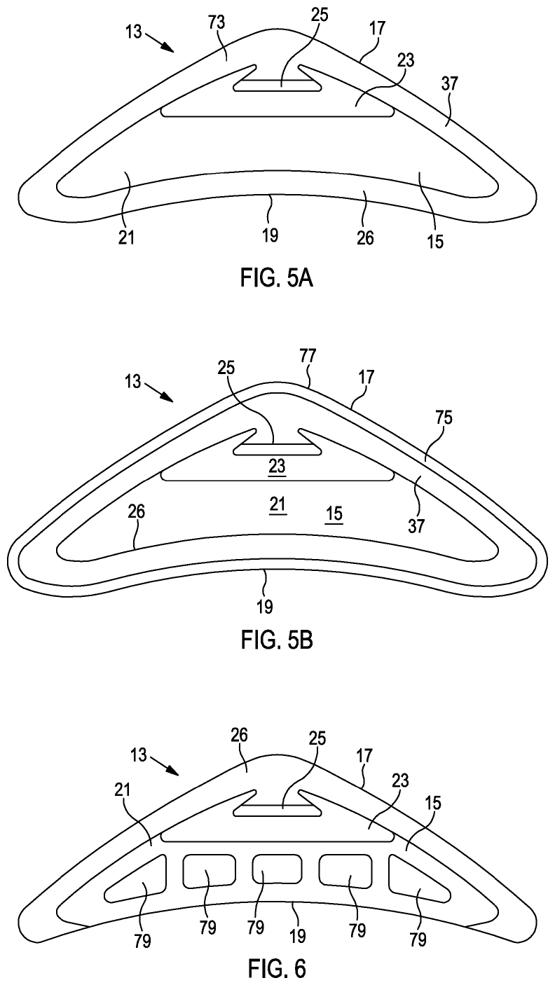

FIG. 5A schematically illustrates a cross section through further example of an ocular prosthesis;

FIG. 5B schematically illustrates a cross section through yet a further example of an ocular prosthesis;

FIG. 6 schematically illustrates a cross section through another example prosthesis;

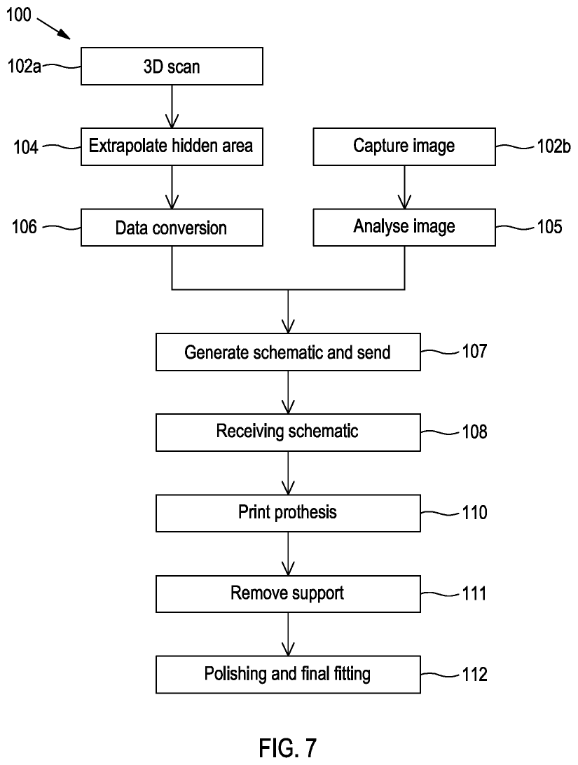

FIG. 7 schematically illustrates a flow chart showing a process of making an ocular prosthesis;

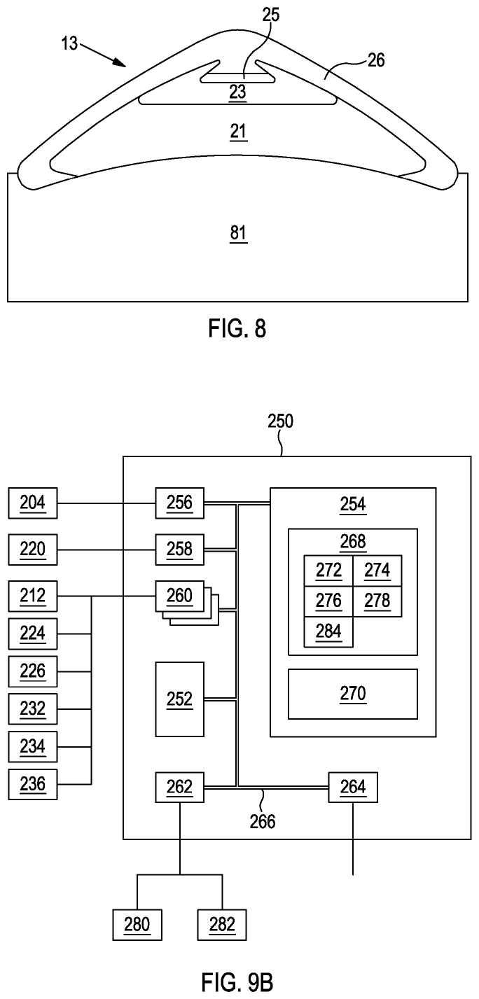

FIG. 8 illustrates an ocular prosthesis printed on a support material;

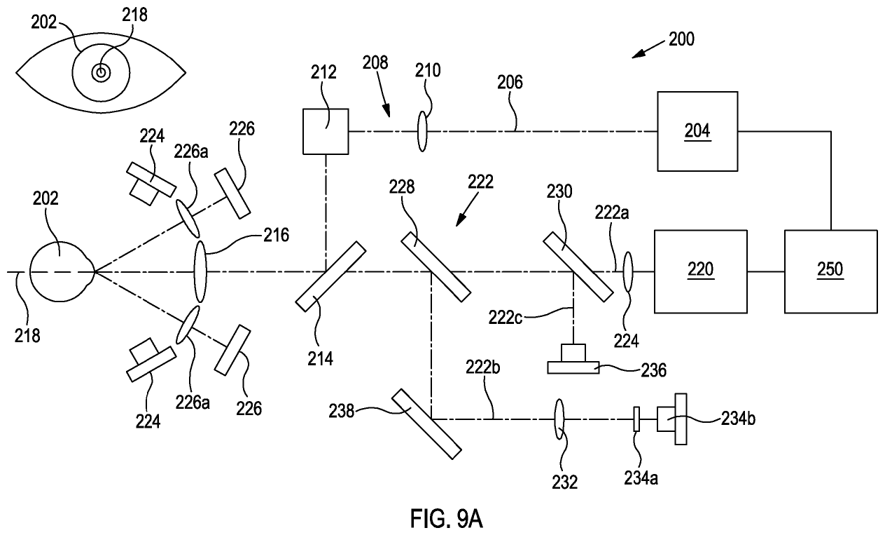

FIG. 9A schematically illustrates a system for simultaneously capturing OCT scans of the ano/micro-phthalmic eye and companion eye, and a colour calibrated image of a companion eye; and

FIG. 9B illustrates an example of control circuitry for the system of FIG. 9A.

FIGS. 2A, 2B and 3A to 3D schematically illustrate an ocular prosthesis 13 according to various embodiments of the invention. The ocular prosthesis 13 may be made by a 3D printing process, such as polyjet printing, although any suitable 3D printing or additive manufacturing technique may be used.

The 3D printing process may be considered to print the prosthesis 13 by printing a plurality of voxels 39 (volumetric pixels) representing a three dimensional volume. As will be discussed below in more detail, each voxel has constant properties (such as colour, hardness and the like) and the variations in properties, such as colour and hardness can be varied by printing voxels differently. For example, colour variation can be achieved by printing different colour voxels. Typically voxels are cubic, but this need not necessarily be the case.

The illustrated prosthesis 13 is formed by a body 15. The body 15 has a convex front surface 17 shown in front on view in FIG. 2A, and an opposing concave rear surface 19 shown in rear view in FIG. 2B. A thickness extends from the front surface 17 to the rear surface 19. The thickness between the front and rear surfaces 17, 19 may vary across the area of the prosthesis 13. FIG. 3A illustrates the body 15 in cross-section. The cross-section is taken through the line 3-3 illustrated in FIG. 2A.

The body 15 includes a number of three dimensional regions 21, 23, 25, 26. Each region 21, 23, 25, 26 has a thickness in the same direction as the thickness of the body 15, and a cross sectional area perpendicular to the thickness.

A first region 21 forms the sclera of the prosthetic eye 1, a second region 23 forms the iris, and a third region 25 forms the pupil. A fourth region 26 forms the cornea of the prosthetic eye 1.

The first region (sclera) 21 is shown in more detail in cross-section in FIG. 3B. The sclera region 21 has a concave rear surface 41, and convex front surface 43. A recess 45 is formed in the front surface 43, with a flat base 47. The recess 45 is substantially circular in shape and extends partway through the thickness of the sclera region 21. Alternatively, the front surface 43 may have a flat spot (not shown) rather than a recess.

The second region (iris) 23 is substantially disc like when viewed from the front (FIG. 2A). FIG. 3C shows the iris region 23 in more detail in cross-section. The iris region 23 has a flat rear surface 49, and a convex front surface 51.

The rear surface 49 of the iris region 23 sits on the base 47 of the recess 45 in the sclera region 21. The shape of the convex front surface 51 of the iris region 23 is arranged to align and continue the convex front surface 43 of the sclera region 21, as shown in FIG. 3A.

At the centre of the iris region 23, a recess 53 is formed. The recess 53 extends partway through the thickness of the iris region 23, and is substantially circular in shape, with a flat base 55. The cross sectional area of the recess 53 increases towards the base 55, such that an overhanging portion 57 is formed.

The pupil region 25 is formed at the base 55 of the recess 53, but does not extend the full thickness of the recess 53. The overhang portion 57 of the iris region 25 overhangs a peripheral edge 59 of the pupil region 25.

The cornea region 26 forms the entire front surface 17 of the prosthetic eye. In cross-section, the cornea region 26 is shaped to form a corneal bump 61 on the prosthesis 13. The cornea region 26 ensures that when viewed from the front, the appearance of the prosthesis 13 is realistic. Furthermore, the optical properties of the cornea region 26 ensure the cornea region 26 mimics the lensing of a real eye, providing the proper effect for the iris and sclera patterns.

In the example shown, the cornea has a front portion 63 extending fully over the front 43 of the sclera region 21, and the front 51of the iris region 23, and has a rear portion 65 extending partway onto the rear of the sclera region 21. Therefore, the majority of the rear surface 19 of the prosthesis 13 is formed by the rear surface 41 of the sclera region 21, and an outer portion 67 of the rear surface 19 is formed by the cornea region 26.

The shape and size of the body 15 is arranged to sit in the space formed between an orbital implant 9 and the eyelids 27. In particular, the rear surface 19 of the body is shaped to conform to the surface of the implant 19, when covered by the conjunctiva 11, and the front surface 17 is shaped to sit behind the eyelids 27.

As a result of the 3D printing process, the body 15 may be considered to be formed of a plurality of layers 29, as illustrated in cross-section in FIG. 3D. In the example shown, the layers 29 are planar layers of equal thickness. Each layer 29 extends in a plane substantially perpendicular to an axis A formed normal to the turning point in the front surface 17 of the prosthesis, and parallel to each other layer 29 (i.e. the layers are parallel to the cross-sectional area of the regions 21, 23, 25, 26).

Each layer 29 in the prosthesis 13 is the height of a single voxel 39. Typically the layers 29 are between 10 and 30 microns thick. In one example process, the layers 29 may be between 14 and 27 microns thick. In a specific example, the layers 29 are 14 microns thick.

As discussed above, the front and rear surfaces 17, 19 are convex and concave surfaces respectively. Therefore, the front and rear surfaces 17, 19 extend through a plurality of layers 29. As such, as best shown in FIG. 3D, the rear surface 19 of the ocular prosthesis 13 may be formed of exposed portions 31 of a number of layers 29 towards the rear of the prosthesis 13 and the front surface 17 is formed of exposed portions 33 of layers 29 near the front of the ocular prosthesis 13.

Typically, the overall envelope 89 of the prosthesis 13 (i.e. the small possible cuboid that the prosthesis can fit within) has a thickness (in the same direction as the thickness of the prosthesis 13) of between 5 and 15 mm. In one typical example, the thickness may be 16 mm. The cross-sectional area of the envelope is typically square, and may have an area between 12 mm by 12 mm and 35 mm by 35 mm. In one example, the envelope may have an area of 25 mm by 25 mm. It will also be appreciated that in some examples, the area of the envelope may be rectangular, with each side having a length between 12 mm and 35 mm.

The envelope 89 is illustrated in FIGS. 2B and 3A for reference. It will be appreciated that due to the shape of the prosthesis 13, no part of the body 15 is ever as thick as the thickness of the envelope 89.

Within the prosthesis, the thickest part of the pupil region 25 and cornea region 26 is along the axis A (see FIG. 3D) defined in the direction of the thickness. For the iris region 23, the thickest part is defined along the same direction, however, the thickness is measured to an imaginary apex formed in the iris region 23, assuming the recess 53 is not included. For the sclera region 21, the thickest part is along an axis at 45 degrees to the axis A in FIG. 3D. In all regions, the thickness may taper towards the outer periphery of the prosthesis 13.

The thickest part of the sclera region may be between approximately 1 mm and 18 mm. The iris region 23 may be between approximately 0.25 mm and 2 mm thick at its thickest point. The pupil region 25 may between 0.25 mm and 1.5 mm thick at its thickest point. The transparent cornea region 26 may be between approximately 1 mm and 5 mm thick at its thickest point, at the corneal bump 61.

By forming the body 15 using a 3D printing process, the body is formed as a single continuous structure. In other words, the body 15 is formed of a single unitary piece. This means that the separate regions 21, 23, 25, 26 are formed together, with no structural joints or weaknesses between them. Similarly, the layers 29 are formed without structural joints or weaknesses between them, preventing delamination.

As discussed above, each of the regions 21, 23, 25, 26 of the body 15 are three dimensional, with a thickness. As such, each of the separate regions 21, 23, 25, 26 is formed by multiple layers 29. It may, therefore, be considered that each region 21, 23, 25, 26 is formed of a number of portions, each portion defined in a single layer. Furthermore, the pattern in each of the sclera, iris and pupil regions 21, 23, 25 is defined into the thickness of the body 15, and not just on the respective front surfaces 43, 51 of the regions or the front surface 17 of the body 15.

In some cases, a single layer may have portions of more than one region 21, 23, 25, 26. For example, the layer labelled 29a in FIG. 3D includes portions of the cornea region 26, iris region 23 and pupil region 25. Similarly, the layer labelled 29b includes portions of the cornea region 26, sclera region 21 and iris region 23. Conversely, other layers, such as the one labelled 29c may only have a portion of a single region, such as the cornea region 26.

Patterns of the different sclera, iris and pupil regions 21, 23, 25 are formed by adding different dyes or pigments to the material of the body 15 in different areas. The sclera, iris and pupil regions 21, 23, 25 are patterned such that when viewed from the front, as in FIG. 2A, the prosthesis forms the appearance of a healthy eye. The front surface 51 of the iris region 23, and the front surface 43 of the sclera region 21 are visible through the transparent cornea region 26 to form the patterns. Furthermore, at least a central part 69 the pupil region 25 is visible through the recess 53 in the iris region 23. A peripheral edge 71 of the front view is formed by the cornea region 26, where it extends over the edge of the sclera region 21.

The pattern in the sclera region 21 is generally an off white colour forming a background. Sub-regions 21a may be formed which define veins and other features formed within the sclera. The background colour and the colour of the other sub-regions 21a may vary across the area of the sclera region 21.

The pattern in the iris region 23 may include various patterns such as whirls, lines, spots and other features. Furthermore, the colour may again vary across the area of the iris region 23. As will be appreciated, there is a wider variety of colours in the iris region 23 than the sclera region 21 or the pupil region 25.

The pupil region 25 is generally dark with limited or no features. This may be formed by a black dye or ink.

Each of the sclera, iris and pupil regions 21, 23, 25 is a three dimensional region, having an area and a depth extending at least partway through the thickness of the body 15. Through the layers 29, the pattern in the regions 21, 23, 25, is varied to provide depth to the final appearance. This may include varying the colour and/or other patterns. For example, the pattern in the layers 29 of the iris region 23 may be varied through the layers 29, and the background colour of the sclera region may be varied through the layers.

In order to allow the depth of the layers to build up a three dimensional pattern, when viewed from the front, each individual layer may be partially transparent, allowing the depth of the pattern to be seen.

As discussed above, the sclera region 21 may include sub-regions 21a defining, for example, veins. The sub-regions 21a are three dimensional sub-regions having a depth that may extend one or more layers through the sclera region 21. The veins may be formed on three different ways:
Formed entirely forward (i.e. towards the front surface) of a front of the sclera region 21. In this case, the vein completely projects on the front of the sclera region 21, and is surrounded by the cornea region 26 on all sides apart from the side joining the view to the sclera region;
Formed entirely within the sclera region 21, such that the vein is surrounded on all sides by the sclera region 21; and
Formed such that the thickness of the vein is partly within the sclera region and partly projecting forward of the sclera region 21, such that the vein is formed at the boundary between the sclera region n21 and cornea region 26.

A prosthesis 13 may have veins of only a single type, or two or more of the type as discussed above. Furthermore, along the length of a vein, the vein may change position within the thickness of the prosthesis 13, such that it varies between the different types along its length.

Similar sub-regions (not shown) may be formed in the iris 23 and pupil regions 25.

As shown in FIG. 3A, the iris and pupil regions 23, 25 only extend a portion of the thickness of the body 15. The sclera region 21 extends behind the rear face 49 of the iris region 23, and forms the majority of the rear surface 19 of the body. The pattern in the sclera region 21 may be continued in layers towards the rear surface 19 of the body 15. Alternatively, at a predetermined boundary, where the material of the sclera is no longer visible from the front, through the layers 29 of the body 15, the sclera pattern 21 may stop, and a further backing pattern provided.

The ocular prosthesis 13 is preferably made of a biocompatible material such as poly(methyl methacrylate) (PMMA). Alternatively, alternative acrylic materials could be used. For example, the VERO™ range of materials, supplied by Stratasys® could be used, or MED 610. The VERO™ range of materials includes different colour materials such as: VERO Cyan, Vero Magenta, VERO Yellow, VERO Black, VERO White, VERO clear. Each of these comprise the VERO material with different dyes added. Therefore the different VERO materials, or mixtures thereof, can be used to provide the different colours.

Some materials, such as MED 610 and VERO™ clear, are transparent, and thus suitable for use in the cornea region 26.

VERO is an acrylic compound comprising a mixture of components including (but not limited to) an acrylic monomer; isobornyl acrylate; pheno,4,4′-(1-methylethylidene)bis-, polymer with (chloromethyl)oxirane,2-propenate; diphenyl-2,4,6-trimethylbenzoyl phosphine oxide; titanium dioxide, acrylate acid ester; propylene glycol monomethyl ether acetate; and phosphoric acid.

MED 610 is a mixture of components including (but not limited to) Exo-1,7,7-trimethylbicyclo[2.2.1]hept-2-yl acrylate; tricyclodecane dimethanl diacrylate; bisphenol A epoxy acrylate oligomer; and 2,4,6-trimethylbenoyldiphenylphosphine oxide.

As discussed above, the pattern in at least some of the regions 21, 23, 25 of the ocular prosthesis 13 can be varied both across the area of the regions, and through the thickness of the regions. In other examples, various other properties of the material may be varied in the same way.

For example, in a further embodiment, the hardness of the material may be varied between layers 29 and also within layers 29.

By varying the hardness of the material so that the area towards the rear surface 19 is of softer composition than other parts of the body 15, it can be made so the friction fit between the ocular prosthesis 13 and the orbital implant 9 provides a strong grip. This ensures that when the orbital implant 9 moves, the ocular prosthesis 13 moves in a corresponding manner.

Instead of or as well as making the rear surface of softer composition, varying the the hardness of the material so that the area towards the front surface 17 is harder than other parts of the body 15 allows the front surface 17 to be polished more easily.

In one example, the hardness of the ocular prosthesis 13 may gradually increase from the rear surface 19 to the front surface 17. In other examples, the body of the prosthesis 13 may be split into sections with different hardnesses. For example, a first section, near the rear surface 19, a second section, near the front surface 17, and a third section in between the first and second. Further sections of different hardness may also be provided and the hardness may be constant or varying in any suitable pattern, within each section. The hardness may also vary across the area of the prosthesis 13, as well as through the thickness.

The different regions 21, 23, 25, 26 may form the different sections. Alternatively, sections may be formed within one of the regions 21, 23, 25, 26 or sections may overlap two or more regions 21, 23, 25, 26. A region may include a part of or a whole one or more of the sections.

The hardness of the prosthesis 13 is varied by modifying the composition of the material through the body 15. The hardness of the material may be varied in any number of suitable ways. For example, additives, which vary the composition of the material, can be used to vary the hardness. For example, TANGO™ or other rubber like materials may be added into the material of the body 15, in varying amounts, to vary the hardness.

TANGO is an acrylic material comprising a mixture including (but not limited to) 2-propenoic acid, 2-[[butylamino)carbonyl]oxy]ethyl ester; isobornyl acrylate; phosphine oxide, phenylbis(2,4,6-trimethylbenzoyl)-; xylenes (o-, m-, p-isomers); benzoyl alcohol; acrylic acid ester; carbon black; dipentene; propylene glycol monomethyl ether acetate; ethylbenzene; n-butyl acetate; isomyl acetate; citral; 2,-Di-tert-butyl-p-cresol; and geraniol.

TANGO, when used as an additive, reduces the hardness of VERO or MRED 610. Therefore, in one example, where the front surface 17 is the hardest part of the body 15, and the rear surface 19 is the softest, the material at the front surface 17 may have the lowest proportion of TANGO in the body (or no TANGO) and the material at the rear surface 19 may have the highest.

In other examples, different additives that increase or decrease the hardness may be used. It will be appreciated that the composition of the material may be varied to provide the desired hardness.

FIGS. 4A and 4B schematically illustrates an ocular prosthesis 13 another example of an ocular prosthesis 13. The ocular prosthesis 13 shown in FIGS. 4A and 4B is the same as the prosthesis 13 discussed above, unless states otherwise, and so like reference numerals are used.

In the example shown in FIGS. 4A and 4B, a groove 35 is formed in the rear surface 19 of the prosthesis 13. The groove 35 provides a channel to carry tears across the back surface of the prosthesis 13.

The groove 35 generally runs from a top corner of the rear surface 19 to the diagonally opposite bottom corner. Tears are provided to the human eye through a lacrimal gland in the top outer corner of each eye. Tears flow across the eye, to be collected at the two lacrimal canals positioned in the bottom inner corner of the eye. The ends of the groove 35 are positioned to replicate this function.

Whilst in a healthy eye, tears are provided to the front of the eye, the groove 35 provides a flow of tears to the rear surface 19 of the prosthesis. For an anophthalmic patient, the tearflow is provided to the interface between the rear surface 19 of the prosthesis and the conjunctiva 11. The groove 25 may also be shaped to allow the tearflow to be spread over at least part of the rear surface 19, thus providing lubrication of the prosthesis 13, whilst also ensuring regular flow of tears.

In the example shown in FIG. 4A, the groove 35 has a number of branches 35a,b. However, it will be appreciated that any suitable path (including, for example spirals, curves, straight sections and the like) may be formed. Separate branches 35a,b need not necessarily be provided or any number of branches 35a,b may be provided. Furthermore, there width and/or depth of the groove 35 may increase at one or both ends.

In some examples, a first groove 35 (or set of grooves 35) may be provided near the lacrimal gland and one or more second grooves 35 may be provided near the lacrimal canals. The first and second grooves 35 may not be connected across the rear surface 19. Instead the tears may be carried across the surface by the existing shape of the surface 19. Any number or shape of channels 35 may provide this function.

It will be appreciated that in some cases, the channels may be formed by closed passages within the body 15. Such passages may have openings to the rear surface 19 to provide tearflow. Only part of the channels may be provided in this way, or the whole channel.

FIG. 5A schematically illustrates a further example of an ocular prosthesis 13. Unless otherwise stated, the ocular prosthesis 13 shown in FIG. 5A is the same as the prosthesis 13 discussed above, and so like reference numerals are used.

In the embodiment shown in FIG. 5A, the ocular prosthesis 13 has an outer shell 37 encasing the body 15.

In the example shown in FIG. 5A, the outer shell 37 is formed by the cornea region 26 extending all the way round the sclera, iris and pupil regions 21, 23, 25. However, it will be appreciated that the outer shell 37 may be distinct from the cornea region 26, such that the sclera, iris, pupil and cornea regions 21, 23, 25, 26 are all encased by the shell 37.

At least the front portion 73 of the shell 37 is transparent, such that the pattern formed by the sclera, iris and pupil regions 21, 23, 25 is visible.

In one example, the shell 37 is formed by the same single unitary structure as the rest of the body 15. Thus the shell 37 is formed at the same time as the rest of the body 15, with no structural weakness of joint between the shell 37 and the rest of the body 15. Alternatively, the shell may be formed separately, for example by a dipping process. In this case, the shell may be of materials such as PMMA.

The shell 37 may have a thickness of at between 200 and 500 microns.

The shell 37 may only partially encase the body 15. In one example, where the rear surface 19 is made of softer material than the rest of the body 15, the rear surface 19 may not be enclosed by the shell 37. Alternatively, the hardness of the shell 37 may be varied in the same way as the material of the body 15.

In one example, the coating 37 may be a biocompatible material. Encasing the body 15 in a biocompatible coating allows the material of the rest of the body 15 be made from non-biocompatible components.

FIG. 5B illustrates a further example of an ocular prosthesis 13. Unless stated otherwise the ocular prosthesis 13 shown in FIG. 5B is the same as the prosthesis 13 of FIG. 5A, and so like reference numerals are used.

As with the example shown in FIG. 5A, the prosthesis shown in FIG. 5B includes an outer shell 37, formed by the cornea region 26. In this example, a further coating 75 is applied on the outer surface 77 of the shell 37. The coating 75 may be a hydrophilic material, such as silica. In some examples, the coating may be applied by plasma coating techniques, or similar.

In the example shown, the coating 75 extends around the full extent of the prosthesis 13, however, in other examples, the coating may only extend around a portion of the prosthesis 13. For example, the coating 75 may be applied to the rear surface 19 only.

The coating has a thickness of 10 microns at most, and is used to provide lubrication. For example, the coating may be between 2 microns and 5 microns thick.

The coating 75 may be used instead of or as well as the tear channels 35. The tear channels should be sized such that the coating can be applied to the surface of the tear channels, without filling the grooves 35.

In other examples, the coating may be provided to help the prosthesis 13 grip the implant 9 (together with or instead of varying the material properties).

It will be appreciated that the coating may be used in examples including a full outer shell 37, as shown in FIG. 5B, or in examples without such a shell 37.

Furthermore, in an alternative example, a plasma treatment may be applied to the outer surface 77 of the shell 75 to modify the polymer of the shell 75 to be hydrophilic near the surface 77, forming the coating. The hydrophilic region may extend 2 to 5 microns deep from the outer surface. This treatment may also be applied to the material of the sclera region 21 and/or iris region 23 and/or cornea region 26 if a separate shell is not provided.

FIG. 6 illustrates yet a further example of an ocular prosthesis 13. Unless stated otherwise, the ocular prosthesis 13 shown in FIG. 6 is the same as the prosthesis 13 discussed above, and so like reference numerals are used.

In the example shown in FIG. 6, the body 15 is formed with a number of voids 79. The voids 79 are three dimensional volumes of empty space within the material of the body 15, thus reducing the overall weight of the prosthesis 13.

In the example shown, the voids 79 are formed in the sclera region 21, however, it will be appreciated that voids may be formed in any region 21, 23, 25, 26 of the body 15, and may overlap one or more region 21, 23, 25, 26.

The embodiments discussed above are given by way of illustration only.

Any suitable biocompatible material may be used for the prosthesis (or non-biocompatible with appropriate coating). Furthermore, any suitable 3D printing method can be used to form the prosthesis 13. In one example, the whole body 15 is formed of the same base material with varying additives as discussed above, in order to vary various properties. In other examples, different regions (or even parts of different regions) may be formed by different materials. Furthermore, different materials may be used to provide different colours, hardness of other material variations.

The arrangement of the regions discussed above is only one example of how the regions may be arranged. For example, in the prostheses 13 discussed in relation to FIGS. 1 to 6, the iris region 21 is a domed disc shape, with a recess 53 at the apex to form the pupil region 25. This may be by way of example only. In other examples, the iris region 21 may be substantially annular in shape, with the pupil formed in the opening in the centre of the region 23. In this example, unlike the example illustrated in the drawings, the iris region 23 does not extend behind the pupil 25.

Similarly, it will be appreciated that in some examples, the sclera region 21 may be an annular-like shape, with an opening in the centre for the iris and pupil regions 23, 25. A further region (not shown) making up the body behind the iris and pupil regions 23, 25 may thus be provided. In this case, the rear surface 19 is formed of sclera region 21, the cornea region 26, and the further region. Any suitable shape interface may be formed between the sclera region 21 and the iris region 23.

The prosthesis 13 may be divided into any number of regions rather than the four regions discussed above. For example, the body may include a core region 87, forming the volume of the body behind the sclera, iris and pupil regions 21, 23, 25. The dashed line in FIG. 3A illustrates an example of the boundary for forming the core region 87. The core region 87 may be formed by a portion of the sclera region 21 which is not visible when viewed from the front, and which, accordingly, has no pattern or colouring. In this case, the core region 87 may form all or part of the rear surface 19 of the prosthesis.

In the examples discussed above, each layer 29 is formed of a planar arrangement of voxels 39. As such, each an edge face of each voxel 39 is parallel to the edge face of its neighbour voxel 39. However, in other examples, the layers 29 may not be planar structures, and may instead be arranged into curved surfaces.

In the example above, the layers 29 also all have equal thickness. In some examples, the size of the voxels 39 (and thus possibly the thickness of the layers 29) may be varied across the prosthesis 13. For example, in areas with small features, smaller voxels may be used to increase the resolution of the printing, whilst in other areas, larger voxels may be used. Also, although the size of the voxel may be the same in all three dimensions, it will be appreciated that this need not necessarily be the case. Furthermore, the voxels need not necessarily be cuboid in shape.

In the example shown in FIG. 2A, the limbus 83 (the transition between the sclera region 21 and iris region 23) is a hard edged circle. This is by way of example only. It will be appreciated that the pattern may include feathering effects around the edge. Similarly, the transition 85 between the iris region 23 and pupil region 25 is also shown as a hard edge, but may also include feathering patterns. The feathering patterns may be provided in a single layer 29 of the prosthesis 13, or may be provided as an effect through the layers 29.

In the examples discussed above, the pattern and optionally the hardness may be varied within and/or between layers 29. It will be appreciated that in some layers 29, only the hardness may be varied. Furthermore, in other examples, only the hardness may be varied across the layers 29 of the prosthesis 13, and the aesthetic appearance of the prosthesis 13 may be provided only on the front surface 17.

In addition to the pattern and hardness other properties of the material may be varied within and between layers 29 and over the area of the prosthesis 13, including, but not limited to, transparency, porosity, material composition, and the like. Furthermore, any one or more properties may be varied.

In the above example, the hardness and/or pattern of body 15, or other properties are changed within or between the layers 29. It will be appreciated that the properties may change gradually or in step changes. At the finest resolution, properties may change on a voxel by voxel basis, but will be appreciated that properties may change at a larger resolution.

It will also be appreciated that features such as varying one or more properties, channels 35, shells 37, coatings 75 and other features discussed above may be provided alone or in combination.

The examples discussed above relate to a prosthesis 13 for use in an anophthalmic eye socket. It will be appreciated that for microphthalmic patients, the prosthesis 13 may be of similar structure, but the thickness may be varied, depending on the size of the microphthalmic eye. In practice, the ocular prosthesis 13 for an anophthalmic patient may have a thickness (envelope thickness) of 1 mm or more. Further, for microphthalmic patients who retain some function in their microphthalmic eye, the pupil may be provided as an aperture through the prosthesis 13, to allow light to pass through the body 15 to the eye. Similar prostheses may also be used for phthisical eyes.

A number of different prostheses 13 have been discussed above. FIG. 7 illustrates one example of a method 100 for producing the ocular prostheses 13.

At a first step 102a, three dimensional scans of both of the patient's eye sockets are taken, to capture three dimensional images of both eye sockets.

The 3scan of the anophthalmic (or microphthalmic) eye socket captures the surface on which the prosthesis 13 is to be fitted. Therefore, for an anophthalmic eye socket, the scan is taken with the orbital implant in place, after any swelling has reduced (in the case of an anophthalmic patient).

The 3scan of the healthy eye (also referred to as the companion eye) scans the internal structure of the eye. In particular, the scan captures the internal structure of the anterior chamber of the companion eye.

From the scan, the 3volumetric shape of the iris, cornea and pupil of the companion eye can be determined, along with the shape of the front surface. This may be output in any suitable format (e.g. an STL file) which provides a point cloud mapping each separate region. The volumetric shape may include measurements such as the diameter and volume of the regions.

The 3scans of the two eyes are then combined to form the shape of the prosthesis 13. The surface scan form the anophthalmic (or microphthalmic) eye provides the shape of the rear surface 19 of the prosthesis 13, whilst the shape and size iris, pupil and cornea regions 21, 23, 25, 26 for the prosthesis 13 are taken from the scan of the companion eye.

In one example, the scan may be obtained by anterior segment optical coherence tomography (OCT), as is known in the art.

For the scan of the anophthalmic (or microphthalmic) eye socket, a focal point is provided for the scan. This may be a mark on the conjunctiva 11 of the eye socket (in the case of anophthalmic patient), or a mark provided on a transparent temporary prosthesis (not shown). The use of the temporary prosthesis allows standard anterior segment OCT techniques to be used. The use of the temporary prosthesis also helps to hold the patients eyelids open.

It will be appreciated that the scan is taken facing towards the patient and captures the front of the eye socket 3. Areas of both eyes will be hidden behind the eyelids 27 of the patient. Without line of sight, the scan is unable to capture this area. In some examples, around 30% of the area of the eye may be behind the eyelids 27. As such, at a second step 104, data for the area behind the eyelid 27 is extrapolated.

It will also be appreciated that the three dimensional data describing the patient's eye socket 3 may be resolved in layers. In other words, that data may represent by points on a fixed grid defined by the resolution of the scanner used to take the measurement. As such, a third step 106 of the method 100 may involve converting the data from grid data to three dimensional printing data.

Optionally, where the resolution of the scanner is different to the voxel size of the 3printing technique used, the conversion step 106 may include an interpolation of the data to modify the resolution to that required by the 3D printer.

In addition to the 3scanning, a two dimensional visual image of the companion eye is taken at step 102b. The image may be output in a RAW file format. At step 105, image analysis is performed to identify:
The background colour of the sclera region;
The pattern of veins and other features in the sclera region; and
The colour and pattern of the iris region.

It will be appreciated that, as with the scan, part of the pattern of the eye, in particular the sclera, may be hidden behind the eyelids. Again, this is extrapolated as necessary.

At step 107 the 3data of the prosthesis to be printed and the colour and pattern data extracted from the two dimensional image are combined into 3D printing data or schematic. To do this, the anterior surface of the companion eye is transposed and aligned with the surface of the ano/micro-phthalmic eye. This then aligns the regions 21, 23, 25, 26 with the desired rear surface 19. The textures and patterns derived from the image analysis are than registered onto the regions 21, 23, 25, 26.

The textures and patterns derived from the two dimensional image analysis are applied to the different regions 21, 23, 25, 26 using known techniques such as UVW mapping.

Further information may be included in the 3D printing data, such as a hardness and/or material composition profile, voids, tear channels and the like. Furthermore, random variations in the patterns may be introduced, to prevent the prosthesis 13 being identical in appearance to the companion eye. Also, the shape or material composition of the cornea region 26 may be modified to ensure the correct lensing effect, by matching that the refractive index of the prosthesis 13 matches that of the cornea of the human eye (1.376).

The printing data includes a 3plan for the prosthesis 13 to be printed. In the case of voxel printing, the possible printing volume may be considered to be a three dimensional cuboid shape, resolved into cubic voxels. The plan includes which voxels to print, and which voxels to leave unprinted. Furthermore, for each voxel to be printed, the data includes information such as: colour, material composition and the like.

After the schematic is generated, it is then sent to the 3D printer. At a next step 108, the schematic is received by a 3D printer, and at step 110, the prosthesis 13 is printed.

When 3D printing an object, the object is often provided on a support material 81, to provide a flat surface on which to form the object, as shown in FIG. 8. In the vicinity of the interface between the support material 81 and the prosthesis, it will be appreciated that the concave shape of the rear surface 19 means that there are some layers that include a portion of the prosthesis 13 and a portion of the support material. During printing, the support material 81 and prosthesis are formed as a single continuous part, with no structural weakness.

The support material 81 is a different composition to the object to be printed. The support material 81 is made of a substance that can be dissolved or washed away in water or another solvent. For example, the support material may be SUP 705, supplied by Stratasys®. SUP 705 is an acrylic compound comprising a mixture of several components, including Acrylic Oligomers; 1,2-Propylene glycol; polyethylene glycol; glycerine; a photo initiator; and acrylic acid ester.

At step 111, the prosthesis 13 is removed from the support. The support material is removed by soaking the prosthesis in a bath of isopropyl alcohol. Agitation, for example magnetic stirring or ultrasonic waves or other vibrations may be used. In one example, the prosthesis 13 is soaked for one hour to remove the support material. Any remaining support material is removed using a hard bristle brush, and the prosthesis 13 is then air dried.

Finally, at step 112, post processing, polishing and final fitting occurs. In some situations, multiple polishing steps may occur. For example, an initial polishing may occur immediately after manufacture, and a final polish may occur when the prosthesis 13 is given to the patient by an ocularist. Small adjustments may also be made to the overall size and shape of the body 15, as necessary, when the prosthesis 13 is given to the patient.

In one embodiment, the post processing may include treatment by tumbling in a tumbling device using cylindrical ceramic abrasive chips and water to remove the striations of the 3D printing process and create a smooth homogeneous surface. The tumbling occurs prior to polishing.

A further post processing step, after final polishing, may be to clean the prosthesis 13. The prosthesis 13 may be cleaned in any suitable solvent such as isopropyl alcohol or distilled water. The cleaning may involve various cleaning stages in different solvents, and the solvent may be changed during the cleaning. As above, each cleaning step may include agitation.

In one example, the cleaning may involve the following steps:
Clean in isopropyl alcohol is for an hour;
Replace isopropyl alcohol and clean for a further hour;
Clean in distilled water is for one hour; and
Replace distilled water and clean for a further hour;

Finally, the prosthesis 13 is washed with hand soap, water and a soft brush and allowed to air dry.

The cleaning process discussed above is explained in further detail in “optimising the biocompatibility of 3D printed photopolymer constructs in vitro and in vivo,” by C G Y Ngan et al, Biomed. Mater 14 035007, 2019. Whilst MED 610 and VERO materials are biocompatible, the biocompatibility may be optimised by the cleaning process, which removes chemicals that may leach out of the prosthesis during use.

In the above examples, the three dimensional volumes of the different regions of the eye are scanned. However, this is by way of example only. Instead, the scan may capture the top surface of the cornea, the top surface of the iris, and the rear surface of the anterior segment of the eye. From these surfaces, the necessary volumes can be determined.

During the scanning of the companion eye, the skin surrounding the eye socket may also be measured. This can be removed from the scan using the front surface of the iris as a cut plane.

Instead of scanning and taking an image of the companion eye, a first scan and image may be taken of the patient's eye before removal. The data from this scan and image can be used instead of the scan of the companion eye. A scan of the same socket (which is now anophthalmic) is then taken after the eye is removed and the orbital implant 9 is fitted, as discussed above.

It will also be appreciated that the step of scanning 102 and the final fitting 112 occur with the patient present. All other steps may occur at any other locations. However, the time taken for the full method may only be a few hours, such that the entire process can be completed whilst the patient stays on site.

The scan data from both eye sockets can be combined in any suitable manner to provide the schematic. Any suitable computer system may be used to carry out these steps, and the processing steps 104, 105, 106, 107 (i.e. the extrapolation of hidden parts, and data conversion). Furthermore, the processing steps 104, 105, 106, 107 may occur at distributed locations or servers. Also, the processing steps may occur in any suitable order. For example, the data conversion may occur before the extrapolation.

In the example shown, the support material is attached to the rear surface 19 of the prosthesis 13, however, it will be appreciated that in some examples, the prosthesis 13 may be attached to the support on any surface.

In addition, either the three dimensional scans of both eye sockets and/or the two dimensional image of the companion eye and/or the three dimensional printing data may be stored in memory, remote from the scanning system, to allow easy reproduction of the prosthesis 13.

It will further be appreciated that the layers need not be formed sequentially. For example, multiple layers may be formed in a first region of the prosthesis, before then moving to a second region. Also, any suitable support may be used as a work surface for forming the prosthesis 13.

The colour of the iris and pupil in the two dimensional image taken of the companion eye may appear different under different lighting conditions. This may result in the ocular prosthesis 13 being printed in colours that look different to the companion eye.

In order to address this, the two dimensional image of the companion eye is taken under controlled lighting conditions. The image is taken in a dark environment, with illumination provided from a calibrated light source.

Colour is generally produced in 3D printers by combining either Red, Blue and Green pigments (RGB) or Cyan, Magenta, Yellow and blacK (CMYK) pigments, in each voxel 39. In addition to controlling the lighting of the two dimensional image, the 3printing can be calibrated to ensure that the printer mixes the constituent colours in the correct proportions to properly replicate the detected colours.

FIG. 9A illustrates a system 200 for capturing a colour calibrated two dimensional image and three dimensional OCT scan of a target eye 202 (either the companion eye or ano/micro-phthalmic eye) at the same time.

The system 200 includes a swept-source OCT unit 204. OCT will be well understood by a person skilled in the art. In general, a source of coherent light, such as a laser, is divided into two or more separate beams. One or more reference beams are directed along a known optical path (reference path, not shown), whilst one or more scanning beams 206 are directed towards a target 202. The beam directed towards the target 202 reflects by the tissue of different parts of the eye. The reflected beams from the reference path and target are combined, and detail about the target 202 can be determined by analysing the interference pattern of the two beams. In one example, the reference beam may have a centre-wavelength of 1 micron, and a sweep width of 70 nm.

The OCT unit 204 includes the coherent OCT light source and reference path (not shown) and emits one or more coherent scanning beams 206 to be directed at the target 202.

The system includes a first optical path 208, along which the OCT scanning beam 206 is directed from the OCT unit 204. The OCT scanning beam 206 is collimated by a lens 210, and then passed through a galvanometer scanner 212, onto a first mirror 214. The first mirror 214 directs the target beam 206 through an objective lens 216, onto the target 202. The reflection of the target beam 206 returns along the same path 208.

The OCT scan provides a 2D scan through at least a portion of the depth of the target 202, along a diametric direction perpendicular to an axis 218 perpendicular to the front surface of the target 202, such that the scan forms a cut-plane. In order to scan the full target 202, the galvanometer scanner is controlled to rotate the target beam to take scans at angular intervals around the axis 218.

The first mirror 214 is splitter such that it reflects the OCT target beam 206 and its reflection, and also permits transmission of light along a second optical path 222 as discussed below in more detail. The first splitter 214 may be a wavelength selective splitter such that light in a first range is reflected, and light in a second range is transmitted (e.g. light with wavelength under 1.3 microns is transmitted, and light with wavelength over 1.3 microns is reflected). Alternatively, the first splitter 214 may be a splitter that transmits and reflects part of the light at all wavelengths. In this case, filters may be provided along the separate optical paths 208, 222 to remove unwanted light.

In addition to the OCT unit 204, the system 200 also includes a camera 220 for capturing a two dimensional visible light image of the target 202, a fixation target 234a and light source 234b, and a positioning light source 236. The camera 220, fixation target and light source 234, and positioning light source 236 are provided along different branches 222a,b,c of the second optical path 222. In one example, the camera 220 is a 1.2 Megapixel camera.
The camera 220 is provided along the main branch 222a of the second optical path 222. The main branch 222a extends from the target 202, through, in turn, the objective lens 216, the first mirror 214, a second mirror 228, and a third mirror 230. Each of the second and third mirrors 228, 230 are also splitters, in a similar manner to the first mirror/splitter 214.

The first, second and third splitters 214, 228, 230 are all arranged at 45 degrees to the transmission of light along the main branch 222a of the second optical path 222, and are selected to transmit the light for the two dimensional image. Therefore, the main branch 222a of the second optical path 222 is straight through the sets of splitters 214, 228, 230.

The first splitter 214 selectively transmits all three branches 222a,b,c of the second optical path 222 and reflects light for the first optical path 208. Therefore, from the first splitter 214 to the target 202, the first optical path 208 and all branches 222a,b,c, of the second optical path 208 overlap.

The second splitter 228 reflects light for the second branch 222b of the second optical path 222, and transmits light for the main and third branches 222a,c. The third splitter 230 transmits light for the main branch 222 only, and reflects light for the third branch 222c. Therefore, from the target 202 to the second splitter 228 all three branches 222a,b,c of the second optical path 222 overlap, between the second and third splitters 228, 230, only the main and third branches 222a,c, overlap, and after the third splitter 230, none of the branches 222a,b,c overlap.

The fixation target 234a and light source 234b are provided along the second branch 222b of the second optical path 222, and are used to help the patient using the system 200 fix their line of sight during scans and image capture, such that the eyeball does not move. The target 234a may be in any suitable form, such as a dot, cross or the like, and is back lit by the light source.

From the fixation target 234, the second branch 222b includes a lens 232 for focusing the image of the target 234a, and a reflecting mirror 238 to direct the second branch 222b at the second splitter 228, where it combines with the other branches 222a,c.

The lens 232 is mounted such that it is moveable along the second branch 222b between the target and the reflecting mirror 238, to vary the focus of the target 234a.

Light from the positing light source 236 is directed along the third branch 222c, directly at the third splitter 230, where it combines with the main branch 222a. The light is directed at the eye, and when the patient is correctly positioned with respect to the system 200, the light is reflected from the corneal bump and detected by line sensors 226. The line sensors 226 may include lenses 226a for focusing and/or filtering of the reflections of the positioning light source 236.

In order to ensure the two dimensional image is colour calibrated, as discussed above, the target is illuminated by a controlled main light source 224. The light source 224, and line sensors 226 discussed above, are arranged around the target 202. The light source 224 may be a white light source with high colour rendering properties. For example, the colour render index of the light source 224 may be 80 or more. By way of example, the colour temperature of the light source may be 5500K, but this is by way of example. In one example, the light source 224 may be a calibrated white light source, such as a D50 light source.

In addition, the transmission bands of each of the splitters 214, 228, 230 are selected to optimise the transmission of reflected light from the calibrated source. The selection of the transmission bands may also provide a filtering function. Furthermore, a correction lens 224 may also be provided immediately before the camera 220 along the main branch second optical path 222, to provide chromatic aberration correction.

The light source 224 and patient are positioned so that any reflection of the light source is within the pupil region of the eye. The two dimensional image may be processed to remove any image artefacts, such as captured reflections of the light source 224. This may be, for example, by editing the image to place black discs over the light or by taking multiple images with the light source at different angles, and overlaying the images.

The optical components discussed above may be provided in a housing (not shown) including an aperture adjacent the target. The housing may also include supports or a rest for a patient to rest their head, and hold the patient stationary whilst scanning. The housing may also include a first section that includes the rest, which is held stationary, and a second section. The second section is moveable relative to the first section, to allow the optical paths of the system 200 to focus and/or to be aligned with either eye of the patient, whilst the patient is stationary. The moment may be in the x, y or z directions. This may be based on, for example, a position of the corneal bump with respect to the optical components, determined by the using the reflection of the positioning light source 236 from the corneal bump. At least some of the optical paths may be provided by optical fibers, if necessary.

In addition to the optical components discussed above, the system 200 may include a controller 250 to operate the system 200. FIG. 9B illustrates the controller in more detail.

The controller 250 includes processing circuitry including a processing unit 252, memory 254, OCT driver 256, camera driver 258, optical system drivers 260, an input/output sub-system 262, and a communications interface 264. The processing unit 252, memory 254, OCT driver 256, camera driver 258, optical system drivers 260, input/output sub-system 262, and communications interface 264 communicate with each other via a system bus 266, in a manner well known in the art.

The memory 254 includes program storage 268 and data storage 270. The program storage includes software code that, when executed by the processing unit 252 controls operation of the system through the drivers 256, 258, 260.

It will be appreciated that although reference is made to a memory 254 it is possible that the memory 254 could be provided by a variety of devices. For example, the memory may be provided by a cache memory, a RAM memory, a local mass storage device such as a hard disk, any of these connected to the processing circuitry 250 over a network connection. The processing unit 252 can access the memory 254 via the system bus 266 and, if necessary, communications interface 264, to access program code to instruct it what steps to perform and also to access data to be processed.

The program code may be delivered to memory 254 in any suitable manner. For example, the program code may be installed on the device from a CDROM; a DVD ROM/RAM (including −R/−RW or +R/+RW); a separate hard drive; a memory (including a USB drive; an SD card; a compact flash card or the like); a transmitted signal (including an Internet download, ftp file transfer of the like); a wire; etc.

It will be appreciated that it is entirely possible that a number of computer systems, processing circuits and/or processing units may be connected in parallel, and/or distributed across a network, in order to provide the functionality of the controller.

The program code section 268 of the memory includes a number of separate modules 272, 274, 276, 278, 284 including program code that controls operation of different parts of the system.

An OCT module 272 controls the operation of the OCT unit 204, including analysis of the interference patterns to determine the structure of the target 202. The OCT module 272 captures a series of two dimensional cut-plane scans through the target 202, and also includes instructions to combine the separate scans from around the axis to determine the structure of the different regions of the eye.

Image capture module 274 controls operation of the camera 220 to capture the two dimensional image of the target. The captured image includes gradation values of the intensity of red green and blue for each pixel. Data storage portion 270 of the memory 254 may include calibration data for converting the detected intensities into a known colour space. The calibration data can be obtained prior to shipping of the device, after installation, or at regular intervals during use.

The calibration data may be obtained by any suitable method. In one example, the calibration data may be obtained by capturing an image of a calibration colour chart illuminated by the light source 224. The calibration chart has a plurality of different colour patches of expected outputs for red, blue and green. By comparing the measured outputs and the expected outputs, the calibration data can be generated. Furthermore, background effects may be removed by capturing an image without illumination. In addition, a white balance adjustment target can be used for white balance adjustment.

Both the OCT module 272 and image capture module 274 may include instructions for auto-focussing the system 200, using known auto-focus methods (e.g. by moving the moveable housing to move the patient).

An optical system control module 276 controls the galvometer scanner 212. Light source 224, fixation target and light source 234a,b, X-Y positioning light source 236 and line scanners 226, and other optical components to enable the OCT scan, image capture and auto-focus to take place. The optical control system module 276 may combine with the OCT module 272 and image capture module 274 during operation, and may also control movement of the moveable part of the housing through an associated driver 260.

A position correspondence module 284 is used to generate correspondence between the positions on the two dimension image of the target 202 and positions in the three dimensional scan.

The correspondence is generated by ray tracing, using correspondence definition data, which is also stored in the data storage portion 270 of the memory 254. The ray tracing traces rays of light from the corneal bump of the eye 202. In addition, when generating the correspondence, the internal parameters of the camera and the positioning of the target eye 202 with respect to the eye can also be incorporated, as can any image distortion.

As discussed above, UVW mapping (or UV mapping) is used to map the colour patterns from the two dimensional image onto the three dimensional printing data. The correspondence data may be used to assist in this process, ensuring accurate mapping of a point in the two dimensional image to the three dimensional image. However, it will be appreciated that the colour may vary, either along the direction of the ray traced from the corneal bump, or in the general direction defined by the axis A (i.e. between different layers 29) even when the two dimensional images shows a single colour for a point.

The correspondence definition data is generated by capturing a two dimensional image and three dimensional scan of a reference structure under the same conditions as the two dimensional image and three dimensional scan of the eye. The reference structure is a three-dimensional structure defining a number of reference points, visible from different angles. By identifying reference points in various two dimensional images and three dimensional scans (either by hand or automatically), the relationship between the two dimensional images and three dimensional scans can be generated.

Like the colour calibration data, the correspondence definition data can be obtained prior to shipping of the device, after installation, or at regular intervals during use.

A system co-ordination module 278 controls overall operation of the system. The system control module controls the system 200 to first capture the two dimensional image of the target 202, and then perform the OCT scan (or vice versa). Such a process may take a few seconds at most. Prior to taking the image and scans of the companion eye, the light source 224 is turned on, and sufficient time given for the pupil to react and stabilise. The time taken to take the scans and image after this should be such that the patient does not blink, and the pupil does not further change size.

The input/output sub-system 262 may be connected to further output device such as a display 280. The input/output sub-system 262 may also be connected to input control devices 282 such as a keyboard, mouse, touchscreen or the like for providing user control over the system. This may enable the user to, for example, position the optical components, manual operate the OCT scan or camera 220, manipulate completed scans and the like.

Instead of or as well as the input devices 282, the controller 250 may be connected to an external control device (not shown), such as a computer. The system 200 may be controlled and monitored from the external control device.

Completed scans may be shown on the display 220. Furthermore, the completed scans may be stored in data storage portion 270 of the memory 254, and optionally the data generated may be output over the communications interface 264, for example over the internet.

The connections between the drivers 256, 258, 260 and the components they control may be wired, wireless or a combination of both.

In use, the patient is positioned to capture the image and scan of a first eye. The moveable part of the system 200 is then repositioned to capture the second eye. Where the target is the ano/micro-phthalmic eye, it may be that only the OCT scan is taken, as the image is not required. However, this is optional, and the image may also be captured.

It will be appreciated that the system discussed above 200 is given by way of example only. Any suitable system may be used to capture the information necessary to provide a prosthesis 13 as discussed in relation to FIGS. 1 to 6 and 8. For example, any suitable OCT system may be used to capture the three dimension scan, and scanning technologies other than OCT may also be used.

In some examples, the eye may not be scanned, and the voxel printing data may also be generated by scanning of an impression of the eye socket 3, if necessary.

Furthermore, any suitably colour controlled environment may be used to capture the two dimensional image of the companion eye. Also, the two dimensional image and scan may be captured by entirely different systems, and the colour control may be omitted or provided in other ways.

In the example discussed above, the patterns of the regions 21, 23, 25 are unique to the particular patient, and are prepared based on an image of the healthy eye, but this need not necessarily be the case. In some examples, a number of predefined patterns may exist for one or more of the regions 21, 23, 25. For those regions, the pattern for the prosthesis 13 being made may be selected as the best match for the companion eye (for example by an operator or by image analysis processes). In one example, the pattern of the sclera region 21 may be selected from one of 24 different recipes (although there may be any number of recipes), whilst the pattern iris and pupil regions 23, 25 is a unique pattern based on the image of the companion eye. In other examples the pupil 25 may also be selected from a list of recipes. In further examples, the iris region 23 may also be selected from a number of predefined options.

In some cases, the prosthetic eye 13 need not be made with a pattern to mimic a real eye or the companion eye, and alternative patterns may be provided (e.g. for aesthetic reasons).

It will be appreciated that the ocular prostheses 13 shown in the Figures discussed above are shown schematically for illustrative purposes only. The relative scales and shape of the ocular prosthesis 13 and regions 21, 23, 25, 26 may be exaggerated to illustrate the concepts discussed.

1. A method of forming an ocular prosthesis comprising:
receiving three dimensional scan data of a patient's eye, the three dimensional scan data including volumetric data of at least an iris region, a pupil region and a cornea region of the eye; and
forming an ocular prosthesis by an additive manufacturing process, the ocular prosthesis having a body with a volume,
wherein the body of the prosthesis includes a three dimensional iris region, a three dimensional pupil region and a three dimensional cornea region, and the volumetric data determines the size and shape of the iris region, the pupil region and the cornea region of the prosthesis.

2. The method of claim 1, wherein the shape of a front surface of the prosthesis is determined by the three dimensional scan data.

3. The method of claim 1 [[, or claim 2]], further comprising: receiving second three dimensional scan data of a fitting surface on which the ocular prosthetic is to be fitted,
wherein a rear surface of the prosthesis is formed so as to confirm to the fitting surface.

4. The method of claim 1, comprising receiving image data including colour and pattern information, and wherein a pattern of the different regions of the prosthesis varies through the thickness of the volume based on the colour and pattern information.

5. The method of claim 1, wherein forming an ocular prosthesis by an additive manufacturing process comprises varying a hardness of the body through the thickness of the body.

6. The method of claim 1, wherein forming an ocular prosthesis by an additive manufacturing process comprises varying a colour and/or pattern of the body, wherein the body is formed by depositing a material forming the body on a support, and wherein the colour of the material is changed by varying a pigment added to the material of the body as it is.

7. The method of claim 5, wherein the body is formed by depositing a material forming the body on a support, and
wherein the hardness of the material is changed by varying the relative proportion of an additive included in the material of the body as it is deposited.

8. The method of claim 1, including: forming one or more channels for directing tear flow to a rear surface of the body, during the additive manufacturing process.

9. (canceled)

10. (canceled)

11. The method of claim 1, comprising: forming an outer shell on an exterior of the body, by the additive manufacturing process.

12.-17. (canceled)

18. A method of manufacturing an ocular prosthesis comprising:
scanning an anophthalmic or microphtalmic eye socket in order to measure a three dimensional fitting surface on which an ocular prosthetic is to be fitted; and
manufacturing an ocular prosthesis by an additive manufacturing process, the prosthesis having a rear surface arranged to conform to the scanned fitting surface.

19. The method of claim 18, comprising:
scanning an anterior chamber of a patient's eye;
analysing the scan to determine the size and shape of the sclera, iris, pupil and cornea of the eye; and
forming the ocular prosthesis with sclera, iris, pupil and cornea regions corresponding to the scanned eye.

20. The method of claim 19, comprising:
combining the scanned surface of the anophthalmic or microphtalmic eye socket with the size and shape of the sclera, iris, pupil and cornea of the scanned eye to form a schematic of the prosthesis; and
manufacturing the prosthesis according to the schematic.

21. The method of claim 18, comprising:
capturing an image of the patient's eye;
analysing the image to determine a colour and pattern of the sclera and iris region; and
forming the ocular prosthesis with a sclera and iris region corresponding to the eye;
wherein the colour and pattern of the sclera and iris region are incorporated into the schematic.

22. (canceled)

23. The method of claim 21, wherein the image is captured in controlled lighting conditions.

24. The method of claim 19 comprising:
capturing an image of the patient's eye;
analysing the image to determine a colour and pattern of the sclera and iris region; and
forming the ocular prosthesis with a sclera and iris region corresponding to the eye;
wherein the colour and pattern of the sclera and iris region are incorporated into the schematic; and
wherein the image and scan of the patient's eye are taken consecutively, using a combined scanner and photography system.

25. The method of claim 20, wherein random variations are introduced into schematic prior to manufacture.

26. The method of claim 18, wherein scanning an anophthalmic or microphtalmic eye socket comprises optical coherence tomography.

27. (canceled)

28. (canceled)

29. The method of claim 26, wherein the scan measures point data spaced by a first spacing, and the method further includes:
converting the scan to have point data spaced by a different spacing.

30. The method of claim 18, comprising:
auto-focussing the scan on a temporary prosthesis received in the anophthalmic or microphtalmic eye socket, wherein the temporary prosthesis includes an auto-focussing point spaced from the surface.

31.-67. (canceled)

68. The method of claim 1, comprising:
auto-focussing the scan on a temporary prosthesis received in the anophthalmic or microphtalmic eye socket, wherein the temporary prosthesis includes an auto-focussing point spaced from the surface.
